(19) United States
(12) Patent Application Publication
(10) Pub. No.: US 2008/0294349 A1
(43) Pub. Date: Nov. 27, 2008

(54) Quantitative Evaluation and Image Analysis of Choroidal Neovascular Membrane and Other Retinal and Subretinal Lesions

(76) Inventor: Nabil M. Jabbour, Morgantown, WV (US)

Correspondence Address: Dusty Gwinn;A & G Intellectual Property Group, LLC, PO Box 4254, Morgantown, WV, 26504-4254, US

(21) Appl. No.: 12/118,445

(22) Filed: May 9, 2008

Related U.S. Application Data
(60) Provisional application No. 60/928,436, filed on May 9, 2007.

(51) Int. Cl. G01N 33/48

(52) U.S. Cl. 702/19

(57) ABSTRACT

The present invention can be a method to allow a user to extract and calculate objective data in a reproducible indicator of the initial evaluation, progression or regression of the activity of choroidal neovascularization or other retinal and subretinal lesions summarized as a single number. The method can be applied to either OCT, FA or both as diagnostic tools depending upon the preference of the user and can be used as the steps of Data Collection, Data and Image Analysis, and Computations. The present invention further provides a user a formula for Approximate Lesion Volume, Flourescein index, the change in the flourescein index, lesion volume index, the active lesion volume change, or the regression factor as well as the severity index. The present invention can also be automated in a program to calculate the desired indexes for the user.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

Choroidal neovascularization (CNV) is the most common and most devastating reason for significant visual loss due to Age-Related Macular Degeneration (AMD) and other similar eye conditions such as histoplasmosis, myopia, etc. AMD is the most common cause of blindness in the elderly population, over age 60, in industrialized nations. To date, there is no curative treatment for CNV. However, many recent optical and pharmacologic treatments have allowed clinicians to offer patients vision-saving and even vision-improving therapeutic options. The options may vary from solo therapy with lasers or pharmacologic agents, to combination treatments, and even radiotherapy or surgery.

To choose among these options, the physician and the patient need an objective set of data showing the risk and benefit of each option. The data is normally based on clinical and experimental studies evaluating treatments using certain diagnostic tests and outcome criteria. Traditional tools to diagnose AMD include Amsler Grid, contrast sensitivity testing, central visual field mapping, focal electro-physiology, etc. The most pertinent components in the professional evaluation for the diagnosis and management of AMD are an eye exam utilizing best corrected visual acuity (VA), fluorescein angiography (FA), and optical coherence tomography (OCT).

The simplest and most efficient evaluation and screening for AMD starts with an eye exam by a trained professional. The main components of this exam include VA and a dilated fundus exam.

A normal eye should be able to see clearly in all ranges of vision, both near and far, thanks to the ability of the human lens to focus or accommodate. With age, the lens becomes stiffer and loses its ability to focus for close-up vision. That's why most people over age 40 require reading glasses. Many people are born with eyes of abnormal size and/or power and hence require glasses for distance vision as well. As these eyes age, they will then require correction for distance as well as for close-up vision, hence the need for “bifocals.” The age of computers and TVs has introduced third and even fourth distances that require adequate correction; hence “trifocals” or “variable glasses” are used. “Best corrected vision” refers to measuring the vision with the best-fitting glasses or contact lenses for the distance tested. The need for correction is not a part of macular degeneration. So, if the vision cannot be corrected to “normal” ranges even with the best correction, then that usually is an indication of a problem which is affecting the eye. The best corrected vision can be obtained for a baseline evaluation and for all subsequent follow-ups for AMD. This makes VA one of the most valuable and useful components of the eye exam.

The inside of the eye is like a dark room with a powerful optical window, therefore the back of the eye cannot be examined without the aid of special optical devices and light. Using light and special lenses, the retina can be viewed and examined. A magnified image of the macula, the center of the retina, is all that is required to diagnose the presence or absence of AMD. Sometimes, this is also all that is needed to decide if the AMD is wet or dry. However, there are cases in which it is hard to determine if mild leakage and/or bleeding are present, especially behind the darkly colored layer of retinal pigment epithelium. In order to evaluate this leakage the other components of the professional evaluation must be utilized.

An FA test requires only two components; a fundus camera which is a camera capable of taking pictures of the back of the eye while looking through the front of the eye and a fluorescein dye which is injected at the time of the test. Since the eye is like a dark room, one of the best ways to look at the back layers is to use a glow-in-the-dark dye in FA. The eye has a transparent optical system; therefore no x-ray is necessary, only regular photography. The glow-in-the-dark dye, sodium fluorescein, is a synthetic form of a vegetable extract in a water solution. This solution is usually injected in the hand or arm. The dye colors the blood and seconds later the blood reaching the eye is “glowing in the dark.” A series of pictures of the back of the eye are taken as the blood makes its normal journey through the different layers in the back of the eye. If there are any abnormal structures of the eye or if there are problems related to the blood supply then the pictures can uncover the details of the abnormalities. An example is an eye with AMD that has grown abnormal channels or blood such as leaky vessels. In this case, the glow-in-the dark fluid will be seen leaking out of those vessels and will allow a professional to identify the area of abnormality. An FA tests allows a retina specialist to confirm the presence of wet AMD, identify the size, shape, location and nature of the abnormal channels of blood, and begin the necessary steps for treatment.

Just as an FA is a dye test without x-ray, OCT is a scan of the back of the eye, without x-ray. The eye has transparent optical media. Therefore, light alone can be used to obtain very detailed cross-sectional images of the different layers of the macula. An OCT can identify the volume, thickness and location of the lesions in wet AMD. Also, the amount of accumulated fluid from leakage can be seen and measured. All of this information is helpful in planning as well as following up on treatment for AMD.

The above-mentioned components of the professional evaluation have proven helpful over the years. To date, however, none of these tools have been able to provide a unified, objective, accurate and reproducible criterion or set of criteria to facilitate the objective evaluation of the disease and the assessment of treatment responses of posterior segment lesions, including CNV. Such an index, or indexes, is a must for the scientific community to navigate successfully through all the new developments for providing patients with treatment options. The creation of such indexes is also a must in clinical application as patients are evaluated for and treated with different therapeutic options.

### BRIEF SUMMARY OF THE INVENTION

The present invention can be a method to allow a user to extract and calculate objective data in a reproducible indicator of the status of progression or regression of the activity of age-related macular degeneration represented as a number.

The method can be applied to risk factors as well as diagnostic tools, specifically either OCT, FA, or both, depending upon the preference of the user.

The disclosed method is further made up of the steps of Data Collection, Data and Image Analysis, and Computations.

The present invention further provides a user a formula for Approximate Lesion Volume and provides a user a formula for a Severity Index, summarizing the risk factors.

In addition, the method provides formulas to compute the Flourescein index.

Further, the method provides for a computation of the change in the flourescein index.

The disclosed method can also be used to calculate the change in lesion volume index, the active lesion volume change, or the regression factor.

The present invention can also be automated in a program to calculate the desired index for the user.

The automated program can include the indexes of lesion thickness, lesion volume, Flourescein activity index, change in activity, lesion volume, active lesion volume, or regression factor.

### BRIEF DESCRIPTION OF THE SEVERAL VIEWS OF THE DRAWING

FIG. 1 is a example of the Severity Index.

FIG. 2 is a background measurement for FA Data and Image Analysis.

FIG. 3 is a tracing of the OLB in the FA Data and Image Analysis.

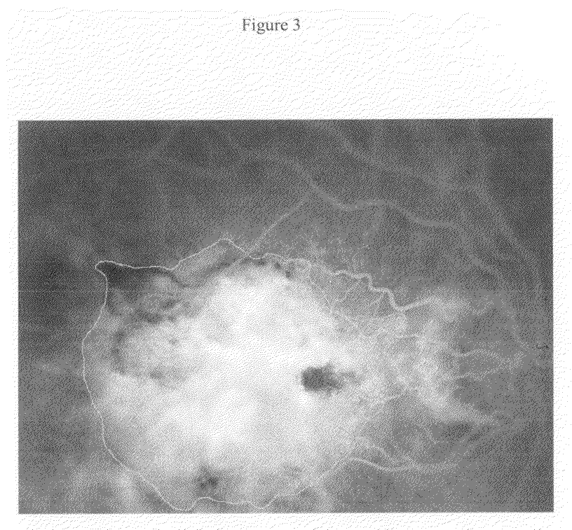

FIG. 4 is an optical density measurement in the FA Data and Image Analysis.

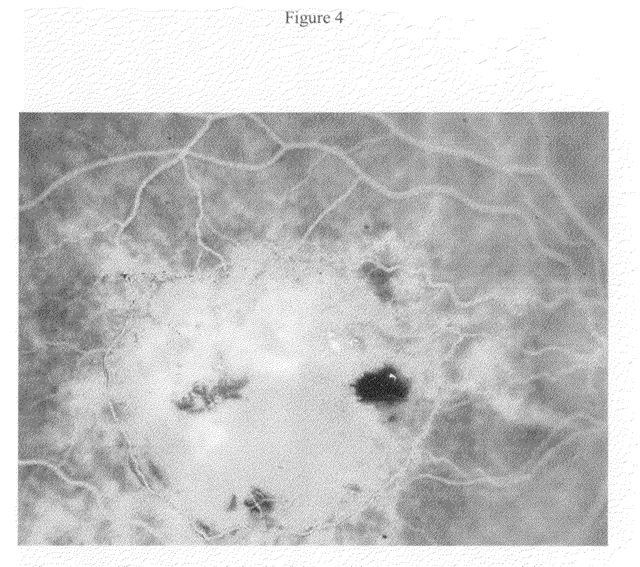

### DETAILED DESCRIPTION OF THE INVENTION

The disclosed method will allow a user to extract and calculate objective data in a reproducible indicator of the initial evaluation, as well as the progression or regression of the activity of choroidial neovascularization or other retinal and subretinal lesions summarized as single numbers. The method can be applied to OCT and FA. Both diagnostic tools are applied in the same steps of Data Collection, Data and Image Analysis, and Computations.

OCT:
1. Data Collection.
   A. Severity Index (SI)
      The severity index is a compilation of seven risk factors. Each factor is graded as 1, 2, 3, or 4 in ascending order of severity.
      Therefore, the least severe lesion will have an SI=7, and the most severe lesion will have an SI=28.
      The seven risk factors and how they are graded are shown in the table below. All evaluations are done within the “circle of interest” defined as the circle drawn with the foveola as center and the distance to the temporal edge of the optic disc as radius (see figure).
      The seven factors initials make the acronym “F.L.A.S.H.E.S.”

| | F | L | A | S | H | E | S |
|---|---|---|---|---|---|---|---|
| | Fluid | Location | Atrophy | | | ETDRS | Surface |
| | (presence of) | (of lesion) | (all spots) | Scarring | Hemorrhage | *Vision | Area |
| 1 | Intra- | Sensory | <25% of | <25% of | <25% of | 20/40 or | <1.5 mm² |
| | retinal | retina | area of | COI | COI | better | |
| | | | COI** | | | | |
| 2 | Sub-retinal | Sub-retinal & | 26-50% of | 26-50% | 26-50% of | 20/50 to | 1.6-10 |
| | | pigment | COI | of COI | COI | 20/125 | mm² |
| | | epithelial | | | | | |
| 3 | Sub- | Pigment | 51-75% | 51-75% | 51-75% of | 20/160 to | 10.1-25 |
| | pigment | epithelial | of COI | of COI | COI | 20/325 | mm² |
| | Epithelial | (no sensory | | | | | |
| | | retina) | | | | | |
| 4 | Combined | Pigment | >75% of | >75% of | >75% of | 20/400 or | >25 mm² |
| | | epithelial & | COI | COI | COI | worse | |
| | | choroid | | | | | |

*ETDR = Early Treatment Diabetic Retinopathy (vision charts)
**COI = circle of interest

B. OCT is a developing field and different commercial units provide different sets of data. The present invention requires an accurate measurement of the lesion volume. Therefore provisions have been made for all available information on known commercial units as of January 2007. The data collection for OCT can be for the overall thickness and/or volume measurement of an area or specific lesion thickness and/or volume measurement.
2. Data and Image Analysis. For OCT Data and Image Analysis comprises of calculating lesion volume and/or thickness. For commercial units that provide lesion-specific volume and/or thickness measurements, the volume is labeled as “LVₚ” and thickness provided as “LTₚ” For units that provide overall volume and/or thickness measurements, the volume measurement is labeled OV and the thickness as OT. Then, for each particular model the OV and the OT is obtained on a random, but standardized, number of eyes such as consistently using a number within a range such as 10-50 eyes with AMD but no CNV to calculate the mean OV which is called the OVₘ and the mean OT or OTₘ. The user then can calculate lesion volume (LV_{c}) and lesion thickness (LT_{c}) as such: LV_{C}=OV−OVₘ and LT_{C}=OT−OTₘ.

FA

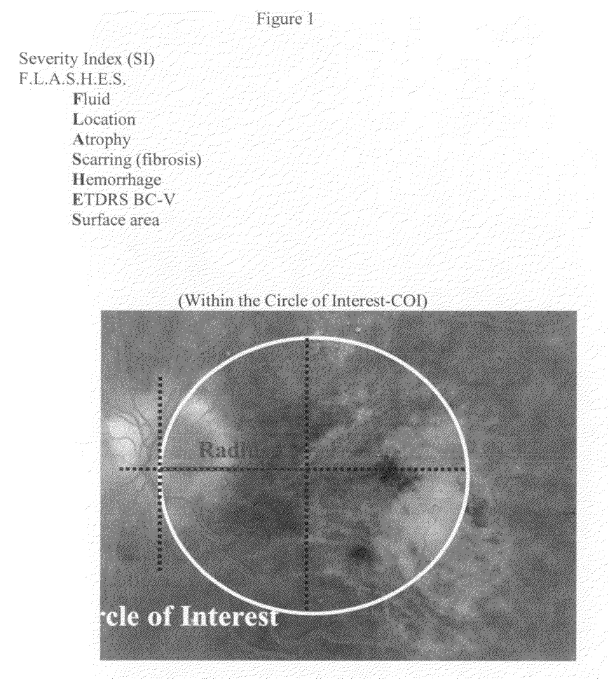

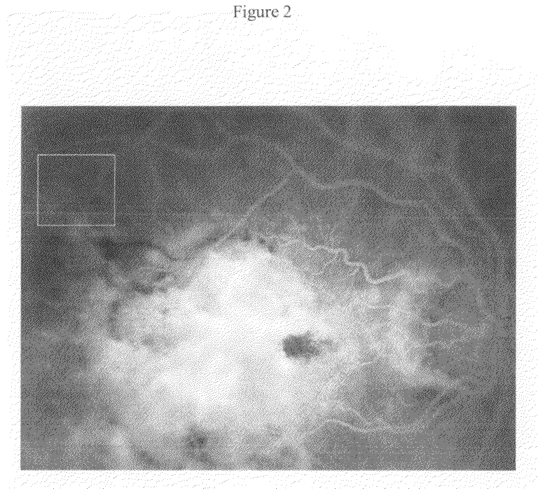

1. Data Collection. Many methods can be used to collect the FA data. Digital black & white, commonly referred to as grey scale, photography has become widely commercially available; however, if film is still being used, the frames of interest need to be digitized. A number of frames such as five standardized frames are obtained for each eye, per visit: f₁—FA image, 1 minute after the injection of dye; f₃—FA image, 3 minutes after the injection of dye; f₅—FA image, 5 minutes after the injection of dye; f_{R}—Red free image, when the image is obtained with the excitation filter on; f_{C}—Control shot, when the image is obtained with both the excitation and barrier filters on. The imaging can be standardized by any one skilled within the art for different time intervals and/or number of frames per visit without undue experimentation.
2. Data and Image Analysis. Using commercial image analysis software like Java the user can use a full frame and a standard square (120×120) away from the fovea for background measurement (B) FIG. 1. Using the 5′ frame of the first FA at the time of the diagnosis (f₀₋₅) where f₀ is an initial visit and each successive visit is numbered consecutively. Using free hand drawing, a user can trace the borders of the lesion and save the outline as OLB which is the original lesion borders. FIG. 2. The user can then run optical density measurements on f₁, f₃, f₅, f_{R} and f_{C} of each date using the 120×120 box for background “Bₓ” where x can be f₁, f₃, f₅, f_{R} or f_{C} and the saved OLB for lesion “Lₓ” where x can be f₁, f₃, f₅, f_{R} or f_{C}. The user can then acquire area measurements on f₅ of every FA study and label SAₓ where x can be the number of visits with the initial visit starting at 0. Data and Image Analysis for FA can comprise surface area (SA) measurements, optical density measurements, and/or borders of lesions.

The final step for either the OCT or the FA version of the method is Computations. There are many ways to calculate the value of fluorescein change using data harvested from the method. The following are just examples.

Approximate Lesion Volume:

For lesions that are closer in shape to a dome, a volume formula

$$V = 1 / 2 A h + \frac{1}{6} 1 / 6 π {h}^{3}$$

where A is floor area and h is height can be used. The approximate lesion volume (LVₐ) can be calculated using the SA for the floor area and the lesion thickness using either LT_{P} or LT_{C} if LT_{P} is not available for height. The formula then can be

$$L {V}_{a} = 1 / 2 ( S A ) ( L T ) + \frac{1}{6} {π ( L T )}^{3} .$$

For lesions that are more like an elliptical shape, the formula can be V= 4/3Ah where A is central area and h is height or ½ thickness. Using SA and T, it can be;

$$L {V}_{a} = \frac{4}{3} ( S A ) \frac{( L T )}{2} = \frac{2}{3} ( S A ) ( L T ) .$$

Flourescein Index (F):

For CNV secondary to AMD a simple formula was found to be sufficient: Fa=(La−Ba)−(LR−BR). For other lesions and diseases, the subtraction indexes and/or ratio values may have to be used as such: Fa=Lna+(La−Ba)−LnR−LnC; or Fa=(Lna−LnR−LnC)+(La−Ba)−(LR−BR)−(LC−BC); or Fa=La−Lr−Ba+BR; or Fa=(La−Ba)−(LR−BR). The flourescein index (Fe) for each eye for each visit can be calculated as Fₓ=F₃+½ leakage index+½ staining index where the leakage index and staining index are defined as: leakage index=F₃−F₁ and staining index=F₅-F₃. Therefore,

$${F}_{x} = {F}_{2} + \frac{F 3 - F 1}{2} + \frac{F 5 - F 3}{2} ;$$

$${F}_{x} = {F}_{2} - \frac{F 1}{2} + \frac{F 5}{2} ; and$$

$${F}_{x} = {F}_{2} + \frac{F 5 - F 1}{2} .$$

The change in flourescein index “C_{F}” can be defined as

$${C}_{F} = \frac{Fx}{Fo}$$

where Fₒ is Fₓ at the baseline visit and x is any study thereafter. So a C=1 indicates no change from baseline
But a C<1 indicates less fluorescence or closure and less activity while a C>1 indicates more fluorescence or growth and more activity. The percentage activity index “A” can be defined as A=(C_{F})(100). Any two visits, other than baseline, can also be compared accordingly.

Change in Lesion Volume Index (Cᵥ).

Cᵥ is a percentage defined as

$${C}_{v} = ( \frac{L {V}_{x} - L {V}_{o}}{L {V}_{o}} ) ( 100 ) ;$$

where LV₀ is lesion volume at base line and where LVx is lesion volume at any visit after that. Depending on which OCT unit is used, the LV could be the LV_{P} when available, the LV_{c} when volume measurements are available and/or the LVₐ when only thickness measurements are available. Active lesion volume change (ΔV) is also a percentage and is defined as ΔV=(Cᵥ)(C_{F}) The regression factor (R) is defined as R=(100−ΔV).

Different indexes will be more valuable for different diagnostic and therapeutic applications. Examples of useful diagnostic indexes are LVa_and SI which could help categorize the disease; Fx could objectively and automatically measures intensity of activity while subtracting artifacts without introducing human bias. In addition, examples of therapeutic indexes are that C_{F} could be used by itself as an objective measure and response to treatment assessing closure and/or could be used to monitor change in activity with or without treatment to decide on course of treatment. C_{V} could be used by itself as an objective measure and response to treatment assessing closure and/or could be used to monitor change in activity with or without treatment to decide on course of treatment. Also, ΔV&R combines therapeutic indexes for more complete and reliable assessment.

Every analysis and computation in this method can be automated into a single computer program which facilitates the acquisition of all of the indexes. One skilled in the art could readily make a computer program that could generate the following indexes: lesion thickness as either LT_{P} or LT_{c}; lesion volume as either LV_{P}, LV_{c}, or LVₐ. Flourescein activity index as Fₓ and the change in activity as C_{F} or A. The change in Lesion volume noted as C_{V} can be CVₐ, CV_{c}, or CVₚ. The active lesion volume change is ΔV and can be ΔVₐ, ΔV_{c} or ΔVₚ. The regression factor is R.

The method can establish data communication between a client and database either via a network or without to perform the above calculations either manually or automatically. The calculations may be implemented using any one of a number of programming languages such as, for example, Matlab, C++, or other programming languages. The network may comprise, for example, the Internet, a local area network, a wide area network, or any other type of network as can be appreciated. The client comprises, for example, a computer system such as a laptop, desktop, or other type of computer system as can be appreciated. In this respect, the client includes a display device, a keyboard, and a mouse. In addition, the client may include other peripheral devices such as, for example, a keypad, touch pad, touch screen, microphone, scanner, joystick, or one or more push buttons, etc. The peripheral devices my also include indicator lights, speakers, printers, etc. The display device may be, for example, cathode ray tubes, liquid crystal display screens, gas plasma-based flat panel displays, or other types of display devices, etc. The client includes a processor circuit having a processor and a memory both of which are coupled to a local interface. In this respect, the client may comprise a computer system or other device with like capability.

The server may comprise, for example, a computer system having a processor circuit as can be appreciated by those with ordinary skill in the art. In this respect, the server includes the processor circuit having a processor and a memory, both of which are coupled to a local interface. The local interface may comprise, for example, a data bus with an accompanying control/address bus as can be appreciated. A number of software components are stored in the memories and are executable by the processors. In this respect, the term “executable” means a program file that is in a form that can ultimately be run by the processors. Examples of executable programs may be, for example, a compiled program that can be translated into machine code in a format that can be loaded into a random access portion of the memories and run by the processors, or source code that may be expressed in proper format such as object code that is capable of being loaded into random access portion of the memories and executed by the processors etc. An executable program may be stored in any portion or component of the memories and including, for example, random access memory, read-only memory, a hard drive, compact disk, floppy disk, or other memory components.

In this respect, the memories are defined herein as both volatile and nonvolatile memory and data storage components. Volatile components are those that do not retain data values upon loss of power. Nonvolatile components are those that retain data upon a loss of power. Thus, each of the memories may comprise, for example, random access memory, read-only memory, hard disk drives, floppy disks accessed via an associated floppy disk drive, compact discs accessed via a compact disc drive, magnetic tapes accessed via an appropriate tape drive, and/or other memory components. In addition, the RAM may comprise, for example, static random access memory, dynamic random access memory, or magnetic random access memory and other such devices. The ROM may comprise, for example, a programmable read-only memory, an erasable programmable read-only memory, an electrically erasable programmable read-only memory, or other like memory device.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

What is claimed is:

1. A method comprising the extraction and calculation of a reproducible indicator of progression or regression of age related macular degeneration further comprising collection of lesion volume data, analyzing one or more of lesion volume and thickness and calculating one or more of a change in lesion volume, change in lesion thickness, change in volume index, active lesion volume change, and regression factor from one or more optical coherence tomography tests.

2. The method of claim 1 further comprising an automation of said collection.

3. The method of claim 1 further comprising an automation of said analyzing

4. The method of claim 1 further comprising an automation of said calculation.

5. The method of claim 1 further comprising an automation of said collection, analyzing, and calculation.

6. The method of claim 1 further comprising the indication of choroidal neovascularization.

7. The method of claim 1 further comprising the indication of retinal lesions.

8. The method of claim 1 further comprising the indication of subretinal lesions.

9. A method comprising the extraction and calculation of a reproducible indicator of progression or regression of age related macular degeneration further comprising collecting fluorescein angiography, collecting one or more of SA measurements, optical density measurements, and borders of legions and calculating one or more of approximate lesion volume, flourescein index, change in flourescein, and change in activity.

10. The method of claim 9 further comprising an automation of said collection.

11. The method of claim 9 further comprising an automation of said analyzing

12. The method of claim 9 further comprising an automation of said calculation.

13. The method of claim 9 further comprising an automation of said collection, analyzing, and calculation.

14. The method of claim 9 further comprising the indication of choroidal neovascularization.

15. The method of claim 9 further comprising the indication of retinal lesions.

16. The method of claim 9 further comprising the indication of subretinal lesions.
